(19) United States
(12) Patent Application Publication
(10) Pub. No.: US 2026/0294760 A1
(43) Pub. Date: Oct. 1, 2026

(54) Bleaching medium containing a photosensitizer

(71) Applicant: WELLA GERMANY GMBH, Darmstadt (DE)

(72) Inventors: Markus SPECKBACHER, Darmstadt (DE); Olga PRYADILOVA, Darmstadt (DE); Mathias HERRLEIN, Darmstadt (DE); Oliver FEY, Darmstadt (DE); Tatjana SCHAEFER, Darmstadt (DE); Ingo WEBER, Darmstadt (DE); Carl-Uwe SCHMIDT, Darmstadt (DE); Alexander THOMYS, Darmstadt (DE); Ioanna-Maria MENEGATOU, Darmstadt (DE); Felix TETSCHNER, Darmstadt (DE)

(21) Appl. No.: 19/489,425

(22) PCT Filed: May 29, 2024
(86) PCT No.: PCT/EP2024/064811

§ 371 Date: Dec. 2, 2025

(30) Foreign Application Priority Data
Jun. 2, 2023 (EP) 23177117.1

(51) Int. Cl. A61K 8/35; A61K 8/22; A61K 8/23; A61K 8/41; A61Q 5/08

(52) U.S. Cl. CPC A61K 8/35; A61K 8/22; A61K 8/23; A61K 8/41; A61Q 5/08; A61K 2800/434; A61K 2800/81

(57) ABSTRACT

The presently claimed invention is directed to a bleaching medium comprising at least one phenalenone which is capable of generating singlet oxygen, at least one persulfate salt, at least one alkalizing agent and at least one hydrogen peroxide source and methods for bleaching hair by using the bleaching medium.

### TECHNICAL FIELD

The presently claimed invention is directed to a bleaching medium comprising at least one photosensitizer which is capable of generating singlet oxygen, at least one persulfate salt, at least one alkalizing agent and at least one hydrogen peroxide source and methods for bleaching hair by using the bleaching medium.

### BACKGROUND

Hair bleaching is a well-established technique in the hair cosmetic industry. Hair bleaching involves the application of an oxidizing agent to the hair for a period that is effective to achieve a desired lighter hair shade. Usually, hydrogen peroxide solution in concentrations ranging from 1 to 12% by weight is the oxidizing agent. The hydrogen peroxide is applied to the hair under alkaline pH conditions and gradually lightens the shade of the hair by oxidizing the melanin that imparts the colour to the hair.

To enhance the lightening efficacy of the peroxide solution, a persulfate salt-based formulation can be incorporated as a “booster”. The sodium, potassium or ammonium persulfate salt is provided in the form of a powder which is mixed with the hydrogen peroxide solution prior to use. The mixed product is then applied to the hair for a period of time that is effective to achieve a desired lighter hair shade.

Currently, hair bleaches are most commonly found in the form of a two-component kit. One component comprises the aqueous hydrogen peroxide composition and the second component comprises a powdered bleach composition that contains persulfate salts and alkaline agents which act as accelerators of the bleaching process, when the two components are combined. This powdered bleach composition may be used alone to bleach hair or in combination with an aqueous hydrogen peroxide composition. The hydrogen peroxide and persulfates are very reactive when combined and form nascent oxygen in addition to hydrogen and sodium sulfide. The nascent oxygen greatly facilitates oxidizing and bleaching of melanin from the hair.

Unfortunately, the process of bleaching hair leads not only to a lightening of the hair, but also to a change in the perceived tone of the hair, leading to what is often described as an off-tone or brassy result, i.e., the hair looks more orange in comparison to untreated hair of similar lightness.

The softness of hair is a highly desirable attribute, contributing significantly to the overall feel and appearance of healthy, well-cared-for hair. Soft hair is generally more manageable, easier to style, and pleasant to the touch, enhancing the aesthetic appeal and comfort for individuals. The softness of hair is often a sign of good hydration and proper conditioning, indicating that the hair cuticles are smooth and well-aligned, reducing friction and tangling. This quality is particularly beneficial as it can reduce breakage and split ends, leading to stronger, more resilient hair over time.

U.S. Pat. No. 9,895,299 B2 describes a process for bleaching or dyeing keratin fibres comprising a step of application of a composition containing one or more chemical oxidizing agents and a step of irradiation of the hair by means of UV-visible radiation.

U.S. Pat. No. 4,792,341 discloses a method and apparatus for artificially bleaching or lightening hair by exposing the hair to intense radiation of artificial light.

EP 451 261 B1 describes a method of bleaching hair under the influence of light, wherein 10 to 120 g of an agent which is either a solution, an emulsion or a gel and contains at least one optical photosensitizer and a compound which is capable of providing a hydrogen radial is applied to hair and is left on hair for up to 80 minutes. Subsequently the hair is irradiated with visible light and/or UV light.

None of the prior art references discloses the use of photosensitizers which generate singlet oxygen for bleaching hair.

There is a need for providing stylists and customers with a bleaching medium that effectively bleaches hair while reducing the level of off-tone and maintaining the level of damage comparable or less to conventional bleaching techniques.

Hence, it is an object of the presently claimed invention to provide a bleaching medium composition that effectively bleaches hair while reducing the level of off-tone and maintaining the level of damage comparable or less to conventional bleaching techniques.

### SUMMARY

Surprisingly, it was found that the addition of at least one photosensitizer which is capable of generating singlet oxygen to a bleaching medium comprising at least one persulfate salt, at least one alkalizing agent and at least one hydrogen peroxide source reduces the level of off-tone which can be effectively formulated into a hair bleaching medium that leads to effective bleaching hair while reducing or maintaining the level of damage to the hair caused by conventional bleaching techniques, as evidenced by a lower combing force, and contributing to the softness of the hair.

Accordingly, in one aspect, the presently claimed invention is directed to a bleaching medium
(A) at least one photosensitizer which is capable of generating singlet oxygen which is at least one phenalenone,
(B) at least one persulfate salt,
(C) at least one alkalizing agent and
(E) at least one hydrogen peroxide source.

In another aspect, the presently claimed invention is directed to a method for bleaching hair comprising at least the steps of:
a) applying the bleaching medium to hair to obtain covered hair,
b) exposing the covered hair to electromagnetic radiation having a wavelength in the range of ≥230 nm to ≤1000 nm, preferably in the range of ≥380 nm to ≤1000 nm, more preferably in the range of ≥380 nm to ≤780 nm, even more preferably in the range of ≥380 nm to ≤490 nm, most preferably in the range of ≥390 nm to ≤430 nm,
c) rinsing the bleaching medium from the covered hair and
d) optionally drying the bleached hair.

In another aspect, the presently claimed invention is directed to the use of at least one phenalenone for bleaching hair by generating singlet oxygen.

In another aspect, the presently claimed invention is directed to a phenalenone which is substituted in position 2 of the phenalenone ring with one organic moiety that is substituted with at least one positively charged nitrogen atom and/or at least one neutral, protonable nitrogen atom and at least one negatively charged functional group.

Other objects, advantages and applications of the presently claimed invention will become apparent to those skilled in the art from the following detailed description.

### DETAILED DESCRIPTION

The following detailed description is merely exemplary in nature and is not intended to limit the presently claimed invention or the application and uses of the presently claimed invention. Furthermore, there is no intention to be bound by any theory presented in the preceding technical field, background, summary or the following detailed description.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

Furthermore, the terms “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the subject matter described herein are capable of operation in other sequences than described or illustrated herein. In case the terms “(A)”, “(B)” and “(C)” or “(a)”, “(b)”, “(c)”, “(d)”, “(i)”, “(ii)” etc. relate to steps of a method or use or assay there is no time or time interval coherence between the steps, that is, the steps may be carried out simultaneously or there may be time intervals of seconds, minutes, hours, days, weeks, months or even years between such steps, unless otherwise indicated in the application as set forth herein above or below.

The term “about”, when used in relation to a weight ratio or a weight percentage, means that a specific value should be understood as meaning a range of ±10%. For example, the weight proportion of a compound of about 1%, means a weight proportion ranging from 0.9 to 1.1%. For example, the weight proportion of a compound of about 2.5%, means a weight proportion ranging from 2.25 to 2.75%.

In the following passages, different aspects of the subject matter are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “preferred embodiment” means that a particular feature, structure, or characteristic described in connection with the embodiment is included in at least one embodiment of the presently claimed invention. Thus, appearances of the phrases “in one embodiment” or “in a preferred embodiment” in various places throughout this specification are not necessarily all referring to the same embodiment but may refer to different embodiments of the presently claimed invention. Furthermore, the features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the subject matter, and form different embodiments, as would be understood by those in the art. For example, in the appended claims, any of the claimed embodiments can be used in any combination.

Furthermore, the ranges defined throughout the specification include the end values as well, i.e., a range of 1 to 10 implies that both 1 and 10 are included in the range. For the avoidance of doubt, the applicant shall be entitled to any equivalents according to applicable law.

For the purposes of the presently claimed invention, ‘% by weight’ or ‘wt. %’ as used in the presently claimed invention is with respect to the total weight of the bleaching medium. Further, the sum of wt. % of all the compounds, as described herein, in the respective components adds up to 100 wt. %.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

The measurement techniques described hereinabove and herein are well known to a person skilled in the art and therefore do not limit the presently claimed invention.

By “user” or “customer” it is meant the person preparing the hair treatment composition. The user is for example a professional hair stylist working in a salon and is different from the subject on whose hair the composition is applied, or the user is identical to the person on whose hair the composition is applied. The mixing can take place in a bowl or within a bottle or even through a mixer placed between the stored individual compositions and the point at which it is dispensed to be used. While two component bleaching mediums are the most commonly found in the market, there are also others which use three or more components.

By “lift” (or “lift power”) it is meant the amount of lightening caused by the bleaching of the natural hair pigment melanin by using the bleaching medium of the presently claimed invention. The amount of lift provided by different hair treatment compositions can be compared by using a human natural dark hair sample (e.g., dark hair of an individual of Chinese descent) and measuring the change of colour achieved following application of the compositions. The change in colour can be measured using well known parameters such as L*a*b* values. A composition can be said to provide a higher lift than another composition, when the resulting L* value or the so-called dL* value (given by L*final−L*initial) measured for a given treated sample of dark hair is higher for that composition than for the other composition, using the same experimental conditions. The denomination Level 2 (herein used interchangeably with “demi-permanent” or “tone-on-tone”) and Level 3 (herein used interchangeably with “permanent”) are commonly used in the hair colour trade to differentiate compositions with medium and high lift.

The softness of hair is determined by several factors, including the hair's moisture content, the condition of the cuticle layer, and the presence of natural oils or applied conditioning agents. Objective measurements of hair softness can be made using various techniques, such as tactile evaluation by trained assessors, mechanical testing for flexibility and pliability, and advanced imaging methods to assess the surface smoothness of hair fibers. Additionally, consumer perception studies often involve panellists who rate the softness of hair after using specific products, providing valuable insights into how different formulations affect this critical quality attribute.

The below materials are being used in the embodiments of the presently claimed invention. The presently claimed invention is described with non-limiting embodiments. The listed ingredients are the preferred features as well as other preferred but non-limiting features of the presently claimed invention.

The measurement techniques described hereinabove and herein are well known to a person skilled in the art and therefore do not limit the presently claimed invention.

In one aspect, the presently claimed invention is directed to a bleaching medium comprising
(A) at least one photosensitizer which is capable of generating singlet oxygen which is at least one phenalenone,
(B) at least one persulfate salt,
(C) at least one alkalizing agent and
(E) at least one hydrogen peroxide source.

The term “photosensitizer” in the context of the presently claimed invention refers to compounds which absorb electromagnetic radiation, preferably visible light, UV light and/or infrared light, and thus produce singlet oxygen.

In an embodiment, the bleaching medium comprises at least one second photosensitizer which is capable of generating singlet oxygen (A2) which is preferably selected from the group consisting of curcumins, flavins, riboflavins, phenoxazines, phenothiazines, phthalocyanines, naphthalocyanines, xanthenes, chlorophyll A, chlorophyll B, porphyrines, coumarins, pyrenes, perylenes, acridine orange and tetrapyrroles.

Phenalenones are known to generate singlet oxygen, cf. e.g., J. Photochem. Photobiol. A: Chem, 79 (1994) 11-17 and New J. Chem., 1999, 23, 85-93.

In an embodiment, the phenalenones are unsubstituted phenalenone or phenalenones which are substituted, preferably which are substituted in position 2 of the phenalenone ring, with at least one organic moiety that is substituted with at least one positively charged nitrogen atom and/or at least one neutral, protonable nitrogen atom and at least one negatively charged functional group.

In an embodiment, suitable flavins are disclosed in EP 2 723 342 A1, EP 2 723 743 A1, EP 2 723 742 A1, and US 2019/0111168 A1.

In an embodiment, suitable curcumins are disclosed in US 2019/0111168 A1. Suitable curcumin derivatives and their manufacture is also described in CA 2 888 140 A1 and suitable curcumin-3,5-dione derivatives and their manufacture are similarly described in EP 2 698 368 A1.

In one embodiment, the phenoxazine is preferably nile blue.

In one embodiment, the phenothiazines are preferably selected from the group consisting of methylene blue, toluidine blue, 1,9-dimethyl methylene blue and methylene green.

In one embodiment, the phthalocyanines are preferably selected from the group consisting of zinc phthalocyanine, aluminium phthalocyanine, zinc phthalocyanine tetrasulfonate and tetrakis(p-trimethylammonium)phthalocyanine zinc chloride.

In one embodiment, the xanthenes are preferably selected from the group consisting of pyronine G, eosine B, eosine Y and rose Bengal.

In one embodiment, the porphyrines are preferably selected from the group consisting of 5,10,15,20-tetrakis(1-methyl-4-pyridinio)porphyrine-tetra(p-toluenesulfonate) and tetrakis(p-trimethylammoniumphenyl) porphyrine chloride.

In one embodiment, the tetrapyrroles are preferably selected from the group consisting of chlorin, chlorin e6 and bacteriochlorin.

In one embodiment, the at least one phenalenone (A) preferably generates singlet oxygen, when exposed to electromagnetic radiation having a wavelength in the range of ≥230 nm to ≤1000 nm, preferably in the range of ≥380 nm to ≤1000 nm, more preferably in the range of ≥380 nm to ≤780 nm, even more preferably in the range of ≥380 nm to ≤490 nm, most preferably in the range of ≥390 nm to ≤430 nm. The most suitable wavelength for the photosensitizer to generate singlet oxygen is selected based on the specific chemical nature of the photosensitizer.

In one embodiment, the at least one phenalenone (A) is preferably present in an amount in the range of ≥0.01 wt. % to ≤10.0 wt. %, preferably in an amount in the range of ≥0.10 wt. % to ≤8.0 wt. %, more preferably in an amount in the range of ≥0.15 wt. % to ≤5.0 wt. %, most preferably in an amount in the range of ≥0.20 wt. % to ≤3.0 wt. %, based on the total weight of the bleaching medium.

In one embodiment, the at least one phenalenone (A) is preferably dissolved in at least one polar solvent before being blended with the other components of the bleaching medium.

The ability of a solvent to dissolve a given substance, e.g. a phenalenone, may conveniently be evaluated by parameter consideration according to the “Hansen system”, which is described in “Hansen Solubility Parameters—A Users Handbook”, published by CRC Press (2000). According to the Hansen system, a solvent or mixture of solvents may be described by three solubility parameters bd (dispersion parameter), δp (polarity parameter) and δh (hydrogen bonding parameter).

In a preferred embodiment, the at least one polar solvent is selected from the group consisting of alkyl esters of fatty acids such as methyl ester of fatty acids ethyl esters of fatty acid and isopropyl ester of fatty acids, such as methyl oleate, methyl palmitate, methyl laurate, isopropyl myristate or isopropyl palmitate and alcohols such as ethoxy diglycol, 2-methoxy ethanol or 1-methoxy-2-propanol and dimethyl sulfoxide and 1-methyl-2-pyrrolidon.

In a preferred embodiment, the at least one polar solvent has Hansen solubility parameters in the range of δd≥15.0 to ≤20.0 (MPa)^{1/2}, δp≥2.0 to ≤28.0 (MPa)^{1/2} and δh≥5.0 to ≤13.0 (MPa)^{1/2}.

In one embodiment, the at least one solvent is preferably present in an amount in the range of ≥0.5 wt. % to ≤5.0 wt. %, preferably in the range of ≥0.5 wt. % to ≤2.0 wt. %, based on the total weight of the bleaching medium.

The bleaching medium contains at least one persulfate salt (B). In one embodiment, the at least one persulfate salt (B) is selected from the group consisting of alkaline earth metal persulfate salts, alkali metal persulfate salts and ammonium persulfate salts that exhibit oxidizing activity, i.e., generating active oxygen, when combined with an aqueous composition comprising hydrogen peroxide. Examples of alkali metal persulfate salts include lithium persulfate, sodium persulfate, potassium persulfate, and caesium persulfate. Examples of alkaline earth metal salts include magnesium persulfate and calcium persulfate. In one embodiment, the at least one persulfate salt (B) is preferably selected from the group consisting of ammonium persulfate, sodium persulfate and potassium persulfate. The persulfate salts are generally present in particulate form, having an average particle sizes in the range from 0.1 μm to 200 μm.

In one embodiment, the at least one component (B) is preferably present in an amount in the range of ≥3.0 wt. % to ≤30 wt. %, more preferably in the range of ≥5.0 wt. % to ≤25 wt. %, even more preferably in the range of ≥7.0 wt. % to ≤20 wt. %, based on the total weight of the bleaching medium.

In a preferred embodiment, the at least one alkalizing agent (C) is selected from the group consisting of ammonia, alkanolamines and inorganic alkalizing agent. In a preferred embodiment, the alkanolamines are selected from primary amines having a C2-C6 alkyl base which carries at least one hydroxyl group. Preferred alkanolamines are selected from the group formed by monoethanolamine, 3-aminopropan-1-ol, 2-amino-1-propanol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol and 2-amino-2-methylpropan-1,3-diol.

In another embodiment, the bleaching medium contains other alkalizing agents, especially inorganic alkalizing agents. Inorganic alkalizing agents usable as contemplated herein are preferably selected from the group consisting of sodium hydroxide, potassium hydroxide, calcium hydroxide, barium hydroxide, sodium phosphate, potassium phosphate, alkali metal silicates, alkali metal metasilicate, alkaline earth metal silicates, alkaline earth metal metasilicate, sodium carbonate and potassium carbonate. In a preferred embodiment, the inorganic alkalizing agents are selected from the group consisting of sodium silicate and sodium metasilicate.

In one embodiment, the bleaching medium comprises at least one alkalizing agent (C) which is sodium silicate.

In another embodiment, the at least one component (C) is present in an amount in the range of ≥3.0 wt. % to ≤20 wt. %, preferably in the range of ≥4.0 wt. % to ≤15 wt. %, more preferably in the range of ≥5.0 wt. % to ≤12 wt. %, based on the total weight of the bleaching medium. The at least one component (C), such as sodium silicate, is an alkaline salt which delivers an aqueous, basic pH, when the bleaching medium according to the presently claimed invention is combined with an aqueous medium.

Since persulfate salts and hydrogen peroxide are relatively stable in acidic medium, it may be necessary to activate them at neutral to basic pH to obtain an adequate formulation. It is thus common practice to add to the bleaching medium at least one alkalizing agent (C) such as alkaline alkali metal silicates and/or ammonium silicates. The basic pH is an activating factor for enabling oxidation by an oxidizing agent such as hydrogen peroxide in combination with persulfate salts.

In one embodiment, the bleaching medium according to the presently claimed invention preferably comprises at least one additive (D) selected from the group consisting of sources of carbonate ions or hydrogen carbonate ions or carbamate ions, aluminosilicates, surfactants, chelants, thickening agents, fillers, amino acids, hydrolyzed proteins, fatty substances, saturated acyclic terpanes having 10 to 40 carbon atoms, C3-C20 monocarboxylic acids and C3-C10 di- or tricarboxylic acids.

In another embodiment, the at least one additive (D) is preferably present in an amount in the range of ≥2.0 wt. % to ≤30 wt. %, more preferably in the range of ≥3.0 wt. % to ≤25 wt. %, even more preferably in the range of ≥4.0 wt. % to ≤20 wt. %, yet even more preferably in the range of ≥4.0 wt. % to ≤18 wt. %, most preferably in the range of ≥4.0 wt. % to ≤17 wt. %, in particular in the range of ≥4.0 wt. % to ≤15 wt. %, based on the total weight of the bleaching medium.

In one embodiment, the sources of carbonate ions or hydrogen carbonate ions or carbamate ions are preferably selected from the group consisting of sodium, potassium, guanidine, arginine, lithium, calcium, magnesium, barium, ammonium salts of carbonate, carbamate and hydrogen carbonate ions. In another embodiment, the at least one source of carbonate ions or hydrogen carbonate ions or carbamate ions is preferably present in an amount in the range of ≥1.0 wt. % to ≤10.0 wt. %, more preferably in the range of ≥2.0 wt. % to ≤8.0 wt. %, even more preferably in the range of ≥2.0 wt. % to ≤6.0 wt. %, based on the total weight of the bleaching medium. The sources of carbonate ions or hydrogen carbonate ions or carbamate ions are solid alkaline salts which deliver an aqueous, basic pH when the bleaching medium is combined with an aqueous medium.

In one embodiment, the aluminosilicate is preferably sodium aluminosilicate. In another embodiment, the aluminosilicates are preferably present in an amount in the range of ≥3.0 wt. % to ≤15 wt. %, more preferably in the range of ≥4.0 wt. % to ≤12 wt. %, even more preferably in the range of ≥4.0 wt. % to ≤10.0 wt. %, based on the total weight of the bleaching medium.

In one embodiment, the bleaching medium according to the presently claimed invention preferably comprises surfactants, more preferably anionic surfactants, which are selected from the group consisting of salts (such as alkaline salts, for example, sodium salts, ammonium salts, amine salts, amino alcohol salts and magnesium salts) of the following compounds: alkyl sulphates, alkyl ether sulphates, alkylamido ether sulphates, alkylarylpolyether sulphates, monoglyceride sulphates; alkyl sulphonates, alkyl phosphates, alkylamide sulphonates, alkylaryl sulphonates, alpha-olefin sulphonates, paraffin sulphonates; alkyl sulphosuccinates, alkyl ether sulphosuccinates, alkylamide sulphosuccinates; alkyl sulphosuccinamates; alkyl sulphoacetates; alkyl ether phosphates; acyl sarcosinates; acyl isethionates; N-acyltaurates; and mixtures thereof. The alkyl or acyl radical of all of these various compounds, for example, comprises from 8 to 24 carbon atoms, and the aryl radical, for example, is chosen from phenyl and benzyl groups. Weakly anionic surfactants can also be used, such as alkyl-D-galactosiduronic acids and their salts, as well as polyoxyalkylenated (C6-C24) alkyl ether carboxylic acids, polyoxyalkylenated (C6-C24) alkylaryl ether carboxylic acids, polyoxyalkylenated (C6-C24) alkylamido ether carboxylic acids and their salts, for example, those comprising from 2 to 50 ethylene oxide groups, and mixtures thereof. Anionic derivatives of polysaccharides, for example carboxyalkyl ether of alkyl polyglucosides, can be also used.

In one embodiment, the surfactants are preferably present in an amount in the range of ≥1.0 wt. % to ≤9.0 wt. %, more preferably in the range of ≥1.0 wt. % to ≤5.0 wt. %, even more preferably in the range of ≥1.0 wt. % to ≤4.0 wt. %, based on the total weight of the bleaching medium.

In one embodiment, the bleaching medium according to the presently claimed invention comprises an anionic surfactant which is preferably selected from the group consisting of alkali metal salts and alkaline earth metal salts of laurate, myristate, palmitate, stearate or behenate, more preferably the anionic surfactant is selected from the group consisting of sodium salts and potassium salts of laurate, palmitate, or stearate.

In one embodiment, the bleaching medium preferably comprises surfactants selected from the group consisting of non-ionic surfactants, amphoteric or zwitterionic surfactants and cationic surfactants.

In one embodiment, nonionic surfactants are selected from the group consisting of fatty alcohol polyglycol ethers, alkylphenol polyglycol ethers, fatty acid polyglycol esters, fatty acid amide polyglycol ethers, fatty amine polyglycol ethers, alkoxylated triglycerides, optionally partly oxidized alk(en)yl oligoglycosides or alk(en)yl polyglycosides or glucuronic acid derivatives, fatty acid-N-alkyl glucamides, protein hydrolysates (particularly wheat-based vegetable products), polyol fatty acid esters, sugar esters, sorbitan esters, polysorbates and amine oxides.

Zwitterionic surfactants are surface-active compounds which contain at least one quaternary ammonium group and at least one —COOH⁽⁻⁾ or —SO₃⁽⁻⁾ group in the molecule. Particularly suitable zwitterionic surfactants are the so-called betaines, such as the N-alkyl-N,N-dimethyl ammonium glycinates, for example, cocoalkyl dimethyl ammonium glycinate, N-acylaminopropyl-N,N-dimethyl ammonium glycinates, for example, cocoacylaminopropyl dimethyl ammonium glycinate, and 2-alkyl-3-carboxymethyl-3-hydroxyethyl imidazolines, containing 8 to 18 carbon atoms in the alkyl or acyl group, and cocoacylaminoethyl hydroxyethyl carboxymethyl glycinate.

Amphoteric surfactants are surface-active compounds which, in addition to a C8 to C18 alkyl or acyl group, contain at least one free amino group and at least one —COOH or —SO₃H group in the molecule and which are capable of forming inner salts. Examples of suitable amphoteric surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group.

Particularly suitable cationic surfactants are quaternary ammonium compounds, preferably ammonium halides, more especially chlorides and bromides, such as alkyl trimethyl ammonium chlorides, dialkyl dimethyl ammonium chlorides and trialkyl methyl ammonium chlorides, for example, cetyl trimethyl ammonium chloride, stearyl trim ethyl ammonium chloride, distearyl dimethyl ammonium chloride, lauryl dimethyl ammonium chloride, lauryl dimethyl benzyl ammonium chloride and tricetyl methyl ammonium chloride.

Hydrogen peroxide is easily decomposed when it comes into contact with heavy metals such as iron, copper and manganese and can quickly become ineffective. This drawback is usually eliminated by the incorporation of chelants into the formulation. In one embodiment, the bleaching medium contains chelants which are preferably selected from the group consisting of ethylenediaminetetraacetic acid (EDTA), ethylenediamine-N,N′-disuccinic acid (EDDS), ethylenediamine-N,N′-diglutaric acid (EDDG), 2-hydroxypropylenediamine-N,N′-disuccinic acid (HPDDS), N,N-dicarboxymethylglutamic acid (GLDA), methylglycine N,N-diacetic acid (MGDA), glycinamide-N,N′-disuccinic acid (GADS), ethylenediamine-N—N′-bis(ortho-hydroxyphenyl acetic acid) (EDDHA), dimethyl glucamine (DMG), N-(1-carboxyethyl)iminodiacetic acid, methylglycine N,N-diacetic acid, diethylenetriamine pentaacetic acid (DTPA), ethylenedicysteic acid (EDC), N,N′-bis(2-hydroxybenzyl)ethylenediamine-N,N′-diacetic acid (HBED), ethylene diamine tri(methylene phosphonate), tetramethylethylenediamine (TMEDA), N,N,N′,N′-tetraethylethylenediamine (TEEDA), and their salts thereof, more preferably the chelants are selected from the group consisting of ethylenediaminetetraacetic acid (EDTA), ethylenediamine-N,N′-disuccinic acid (EDDS), and their salts thereof. By “salts thereof”, it is meant in the context of chelants all salts comprising the same functional structure as the chelant they are referring to and including alkali metal salts, alkaline earth salts, ammonium salts, substituted ammonium salts, and mixtures thereof; alternatively sodium salts, potassium salts, calcium salts, magnesium salts, ammonium salts, and mixtures thereof; alternatively monoethanolammonium salts, diethanolammonium salts, triethanolammonium salts, and mixtures thereof.

In one embodiment, the chelants are preferably present in an amount in the range of ≥0.1 wt. % to ≤3.0 wt. %, more preferably in the range of ≥0.2 wt. % to ≤2.0 wt. %, even more preferably in the range of ≥0.3 wt. % to ≤1.5 wt. %, based on the total weight of the bleaching medium.

The bleaching medium according to the presently claimed invention comprises preferably at least one thickening agent. Thickening agents are typically water-soluble and deliver the desired semi-fluid to pasty consistency to the bleaching medium. Typical thickening agents include, but are not limited to, synthetic water-soluble polymers and water-soluble polymers of natural origin. In one embodiment, the bleaching medium according to the presently claimed invention comprises thickening agents which are preferably selected from the group consisting of cellulose derivatives, a polyacrylic acids, acrylate copolymers, polysaccharide gums, xanthan gums, starch, guars, starch derivatives, alginic acid and acrylic acid-diallyldimethylammonium polymers; more preferably are selected from the group consisting of xanthan gums, starch, and alginic acid.

Exemplary embodiments of water-soluble synthetic polymeric thickening agents comprise polyvinylpyrrolidone, polyacrylic acid, polyacrylamide, non-crosslinked poly-2-acrylamidopropanesulfonic acid, acid crosslinked poly-2-acrylamido-2-methylpropane sulfonic acid, cross-linked poly-2-acrylamido-2-methylpropane sulfonic acid partially neutralized by ammonia. Additional exemplary embodiments of synthetic water-soluble thickening agents include those such as non-crosslinked poly-2-acrylamido-2-methylpropane sulfonic acid in combination with hydroxyalkylcellulose ethers or with poly (ethylene oxide). Embodiments of water-soluble thickening agents derived from natural sources include, but are not limited to, polymers comprising at least one sugar unit. Exemplary embodiments of natural source thickening agents include but are not limited to non-ionic guar gums semisynthetic guar gums modified with C1-C6 hydroxylalkyl groups; biopolysaccharide gums of microbial origin such as scleroglucan or xanthan gums; gums from plant exudates such as arabian, ghatti, karaya, tragacanth, carrageenan, agar and carob gums; pectins; alginates; starches including such examples as rice starch, corn starch, potato starch, canola starch, cassava starch and hydrolyzed derivatives thereof; hydroxyalkyl (C1-C 6) celluloses and carboxyalkyl (C1-C 6) celluloses. Embodiments of water-soluble cellulosic thickening agents include, but are not limited to, hydroxyalkyl (C 1-C 6) celluloses such as hydroxyethylcellulose.

In one embodiment, the thickening agents are preferably present in an amount in the range of ≥0.5 wt. % to ≤5.0 wt. %, more preferably in the range of ≥0.5 wt. % to ≤6.0 wt. %, even more preferably in the range of ≥1.0 wt. % to ≤4.0 wt. %, based on the total weight of the bleaching medium.

In one embodiment, the bleaching medium according to the presently claimed invention comprises fillers which are selected from the group consisting of kaolin clay, smectite clay and a sepiolite clay. In one embodiment, the fillers are preferably present in an amount in the range of ≥0.5 wt. % to ≤10.0 wt. %, more preferably in the range of ≥1.0 wt. % to ≤8.0 wt. %, even more preferably in the range of ≥1.0 wt. % to ≤6.0 wt. %, based on the total weight of the bleaching medium.

In one embodiment, the bleaching medium preferably comprises at least one amino acid selected from the group consisting of alanine, glycine, phenylalanine, leucine, valine, isoleucine, glutamic acid, aspartic acid, citrulline, histidine, arginine, lysine and tyrosine; more preferably at least one amino acid selected from the group consisting of alanine, glycine, valine and arginine; most preferably the at least one amino acid is alanine or glycine or valine or arginine. In a preferred embodiment, the bleaching medium does not contain other amino acids. The at least one amino acid which is present in the bleaching medium of the presently claimed invention is oxidatively stable in a bleaching medium, i.e., the at least one amino acid is not oxidized or only oxidized to a very small degree in a bleaching medium by one of a nucleophilic oxidation reaction, an electrophilic oxidation reaction or a radical oxidation reaction.

In one embodiment, the at least one amino acid is preferably obtained from a natural source, more preferably the at least one amino acid is obtained from a plant source.

In one embodiment, the amino acids are preferably present in an amount in the range of ≥0.05 wt. % to ≤2.0 wt. %, more preferably in the range of ≥0.1 wt. % to ≤1.5 wt. %, even more preferably in the range of ≥0.1 wt. % to ≤1.3 wt. %, yet even more preferably in the range of ≥0.1 wt. % to ≤1.0 wt. %, based on the total weight of the bleaching medium.

In one embodiment, the bleaching medium preferably comprises hydrolyzed proteins which are selected from the group consisting of hydrolyzed keratin and hydrolyzed vegetable protein. Hydrolyzed proteins are commercially available in the market, e.g., AC Vegan Keratin OS, Nutrilan® Keratin LM, Gluadin® Kera-P LM, KeraMatch® V, FK Repair Ultra, FK Restore, FK Protect Plus and ProSina™.

In one embodiment, the bleaching medium according to the presently claimed invention comprises C3-C20 monocarboxylic acids which are preferably selected from the group consisting of propionic acid, butyric acid, pentanoic acid, iso-pentanoic acid, hexanoic acid, iso-hexanoic acid, heptanoic acid, iso-heptanoic acid, octanoic acid, iso-octanoic acid, nonanoic acid, iso-nonanoic acid, decanoic acid, undecanoic acid, dodecanoic acid, tridecanoic acid, tetradecanoic acid, pentadecanoic acid, palmitic acid, heptadecanoic acid, stearic acid, oleic acid, linoleic acid, linolenic acid, nonadecanoic acid and eicosanoic acid.

In one embodiment, the C3-C20 monocarboxylic acids are preferably present in an amount in the range of ≥0.05 wt. % to ≤2.0 wt. %, more preferably in the range of ≥0.1 wt. % to ≤1.5 wt. %, even more preferably in the range of ≥0.1 wt. % to ≤1.3 wt. %, yet even more preferably in the range of ≥0.1 wt. % to ≤1.0 wt. %, based on the total weight of the bleaching medium.

In one embodiment, the bleaching medium according to the presently claimed invention comprises C3-C10 di- or tricarboxylic acids which are preferably selected from the group consisting of tartronic acid, succinic acid, malic acid, maleic acid, citric acid, tartaric acid, adipic acid, glutaric acid, oxalic acid, sebacic acid, glyoxylic acid, malonic acid and hydroxyglutaric acid.

In one embodiment, the C3-C10 di- or tricarboxylic acids are preferably present in an amount in the range of ≥0.05 wt. % to ≤2.0 wt. %, more preferably in the range of ≥0.1 wt. % to ≤1.5 wt. %, even more preferably in the range of ≥0.1 wt. % to ≤1.3 wt. %, yet even more preferably in the range of ≥0.1 wt. % to ≤1.0 wt. %, based on the total weight of the bleaching medium.

In one embodiment, the bleaching medium according to the presently claimed invention comprises at least one fatty substance which is selected from the group consisting of liquid hydrocarbons, non-silicone oils of animal, plant, mineral or synthetic origin, fatty alcohols, fatty acid esters and/or fatty alcohol esters, non-silicone waxes, silicones and saturated acyclic terpanes.

More particularly, the liquid hydrocarbons are selected from the group consisting of:
linear or branched, optionally cyclic, C6-C16 alkanes such as hexane, undecane, dodecane, tridecane, and isoparaffins, such as isohexadecane, isododecane and isodecane,
linear or branched hydrocarbons of mineral, animal or synthetic origin with more than 16 carbon atoms, such as volatile or non-volatile liquid paraffins and derivatives thereof, petroleum jelly, liquid petroleum jelly, polydecenes, and hydrogenated polyisobutene such as Parleam®.

In a preferred embodiment of the presently claimed invention, the liquid hydrocarbons are selected from the group consisting of volatile or non-volatile liquid paraffins, and liquid petroleum jelly.

In a further embodiment of the presently claimed invention, the bleaching medium of the presently claimed invention comprises fatty substances in an amount in the range from 1.0 wt. % to ≤20.0 wt. %, based on the total weight of the bleaching medium.

In an embodiment, the bleaching medium according to the presently claimed invention comprises at least one saturated acyclic terpane which is a monoterpane, a sesquiterpane, a diterpane, a sesterterpane, a triterpane or a tetraterpane. In another embodiment, the at least one saturated acyclic terpane is selected from the group consisting of 2,6-dimethyl octane, 2,6,10,15,19-pentamethylleicosane, farnesane, phytane, squalane, isosqualane, neosqualane and lycopane. In yet another embodiment, the at least one saturated acyclic terpane is selected from the group consisting of squalane, isosqualane and neosqualane, preferably the at least one saturated acyclic terpane is squalane.

As used herein, “squalane” refers to an oil and is also referred to as 2,6,10,15,19,23-hexamethyl tetracosane. In one embodiment, the squalane is synthetic squalane which is obtained by chemical synthesis or semisynthetic squalane which is obtained from naturally occurring squalene by reduction. Squalene is present in deep sea fish, such as sharks, or olive oil, rice bran oil, wheat germ oil, sesame oil and cotton seed oil. Squalane can also be obtained from sugar cane.

In yet another embodiment, the at least one saturated acyclic terpane having 10 to 40 carbon atoms is preferably present in an amount in the range of ≥0.1 wt. % to ≤15.0 wt. %, more preferably in the range of ≥0.5 wt. % to ≤14.0 wt. %, even more preferably in the range of ≥0.5 wt. % to ≤5.0 wt. %, yet even more preferably in the range of ≥1.0 wt. % to ≤4.0 wt. %, most preferably in the range of ≥1.0 wt. % to ≤3.0 wt. %, in particular in the range of ≥1.0 wt. % to ≤2.0 wt. %, based on the total weight of the bleaching medium.

In one embodiment, the bleaching medium preferably comprises at least one hydrogen peroxide source (E) which is selected from the group consisting of hydrogen peroxide, calcium peroxide, urea peroxide, perborates and percarbonates.

The at least one hydrogen peroxide source (E) is preferably hydrogen peroxide, which is preferably present in the range of ≥1.0 wt. % to ≤12 wt. %, preferably in the range of ≥2.0 wt. % to ≤6.0 wt. %, based on the total weight of the bleaching medium.

In one embodiment, the bleaching medium according to the presently claimed invention comprises
(A) ≥0.5 wt. % to ≤3.0 wt. % of at least one phenalenone,
(B) ≥3.0 wt. % to ≤30 wt. % of at least one persulfate salt,
(C) ≥3.0 wt. % to ≤20 wt. % of at least one alkalizing agent,
(D) ≥4.0 wt. % to ≤25 wt. % of at least one additive (D) selected from the group consisting of sources of carbonate ions or hydrogen carbonate ions or carbamate ions, aluminosilicates, surfactants, chelants, thickening agents, fillers, amino acids, hydrolyzed proteins, fatty substances, saturated acyclic terpane having 10 to 40 carbon atoms, C3-C20 monocarboxylic acids and C3-C10 di- or tricarboxylic acids, and
(E) ≥1.0 wt. % to ≤12 wt. %, preferably in the range of ≥2.0 wt. % to ≤6.0 wt. % of at least one hydrogen peroxide source (E), preferably hydrogen peroxide.

In one embodiment, the bleaching medium according to the presently claimed invention comprises
(A) ≥0.2 wt. % to ≤3.0 wt. % of at least one phenalenone,
(B) ≥3.0 wt. % to ≤30 wt. % of at least one persulfate salt,
(C) ≥3.0 wt. % to ≤20 wt. % of at least one alkalizing agent,
(D) ≥4.0 wt. % to ≤25 wt. % of at least one additive (D) selected from the group consisting of sources of carbonate ions or hydrogen carbonate ions or carbamate ions, aluminosilicates, surfactants, chelants, thickening agents, fillers, amino acids, hydrolyzed proteins, fatty substances, saturated acyclic terpane having 10 to 40 carbon atoms, C3-C20 monocarboxylic acids and C3-C10 di- or tricarboxylic acids,
(E) ≥2.0 wt. % to ≤6.0 wt. % hydrogen peroxide, and
≥0.5 wt. % to ≤5.0 wt. % of at least one polar solvent having Hansen solubility parameters in the range of δd≥15.0 to ≤20.0 (MPa)^{1/2}, δp≥2.0 to ≤28.0 (MPa)^{1/2} and δh≥5.0 to ≤13.0 (MPa)^{1/2}.

In one embodiment, the bleaching medium of the presently claimed invention is prepared by mixing a bleach powder comprising the at least one persulfate salt (B) and the at least one alkalizing agent (C) and at least one aqueous medium comprising the at least one hydrogen peroxide source (E), whereby the at least one component (A) can be present in the bleach powder or the aqueous medium or the at least one component (A) is added to the bleach powder or the aqueous medium. In one embodiment, the bleach powder is preferably prepared by dispersing, under mechanical action, all the compounds that are present in particle form, i.e., the at least one persulfate salt (B) and the at least one alkalizing agent (C), in the fatty substances, in which the other liquid components of the bleach powder have been pre-dispersed or premixed.

In another embodiment, the bleach powder is preferably prepared by extrusion, by introducing the liquid and solid phases of the bleach powder into the extruder, and then mixing all components at a temperature s 25° C., using a co-rotating twin-screw system comprising transportation and blending means.

The bleach powder is used in combination with an oxygen donor, such as hydrogen peroxide, to bleach hair.

Thus, in another aspect, the presently claimed invention is directed to a method for bleaching hair comprising at least the steps of:
a) applying the bleaching medium as described herein to hair, which is dry or wet, to obtain covered hair,
b) exposing the covered hair to electromagnetic radiation having a wavelength in the range of ≥230 nm to ≤1000 nm, preferably in the range of ≥380 nm to ≤1000 nm, more preferably in the range of ≥380 nm to ≤780 nm, even more preferably in the range of ≥380 nm to ≤490 nm, most preferably in the range of ≥390 nm to ≤430 nm,
c) rinsing the bleaching medium from the covered hair and
d) optionally drying the bleached hair.

In one embodiment, steps a) to d) can be repeated multiple times, e.g., 2 times or 3 times.

The bleaching medium is preferably applied to the hair with any number of application devices, with the main purpose of scarcely and densely depositing the bleaching medium on the hair to achieve the desired bleaching result.

In one embodiment, the electromagnetic radiation is preferably produced by a source of radiation which is selected from the group consisting of artificial sources of radiation such as UV lamps, IR lamps, fluorescent lamps, light-emitting diodes and lasers.

In one embodiment, the electromagnetic radiation preferably has an energy density in the range of ≥1 mW/cm² to ≤70 mW/cm², preferably in the range of ≥10 mW/cm² to ≤50 mW/cm², even more preferably in the range of ≥20 mW/cm² to ≤40 mW/cm².

In one embodiment, the bleaching medium has a pH of ≤7.0. The acidic pH ensures the stability of the hydrogen peroxide in the bleaching medium. The pH is preferably obtained by the addition of at least one acidifying agent selected from the group consisting of hydrochloric acid, acetic acid, phosphoric acid, lactic acid, malic acid and boric acid. The bleaching medium preferably comprises at least one adjuvant selected from the group consisting of preserving agents, colorants, fragrances, anti-foaming agents, hydrogen peroxide stabilizers, and chelants such as EDTA and EDDS.

Oxidative hair colouring compositions are typically sold in the form of kits comprising individually packaged components such as separate containers, e.g., a dye or tint component comprising a dye coupler and precursors; an aqueous hydrogen peroxide component and a bleaching medium comprising the alkalizing agent and the persulfate salts. Hence, in another aspect, the presently claimed invention is also directed to a kit comprising i) an individually packaged oxidizing component comprising hydrogen peroxide; ii) an individually packaged colouring component comprising at least one dye coupler and at least one dye precursor; and iii) an individually packaged bleaching medium as described herein.

In one embodiment, the hair is bleached by application of the bleaching medium as described herein followed by colouring of the hair by applying colouring components comprising at least one dye coupler and at least one dye precursor. A combination of these procedures allows for using lower amounts of dye couplers and dye precursors in comparison to a procedure comprising conventional bleaching techniques and colouring techniques.

In one embodiment, the dye precursors are selected from of the group consisting of toluene-2,5-diamine, p-phenylenediamine, n-phenyl-p-phenylenediamine, N,N-bis(2-hydroxyethyl)-p-phenylenediamine, hydroxyethyl-p-phenylenediamine sulphate, hydroxypropyl bis(n-hydroxyethyl-p-phenylenediamine), 2-methoxymethyl-p-phenylenediamine, 2-chloro-p-phenylenediamine, p-aminophenol, p-methylaminophenol, 4-amino-m-cresol, 6-amino-m-cresol, bis(5-amino-2-hydroxyphenyl)methane, tetraaminopyrimidine, 2,5,6-triamino-4-pyrimidinol, 4,5-diamino-1-(2-hydroxyethyl)pyrazol 2,3-diaminodihydroxypyrazolopyrazolone dimethosulfonate, 4,5-diamino-1-hexylpyrazol, hydroxypropyl-p-phenylenediamine, dimethylpiperazinium aminopyrazolopyridine chloride hydrochloride, methylimidazoliumpropyl p-phenylenediamine, hydroxyethoxy aminopyrazolopyridine and salts thereof. Preferably, the oxidative primary dye precursors are selected from of the group consisting of toluene-2,5-diamine, 4,5-diamino-1-hexylpyrazol, N,N-bis(2-hydroxyethyl)-p-phenylenediamine, hydroxypropyl-p-phenylenediamine, 2-methoxymethyl-p-phenylenediamine and salts thereof.

In one embodiment, the dye couplers are selected from the group consisting of resorcinol, 4-chlororesorcinol, 2-chlororesorcinol, 2-methylresorcinol, m-aminophenol, 4-amino-2-hydroxytoluene, 2-methyl-5-hydroxyethylaminophenol, 3-amino-2,6-dimethylphenol, 3-amino-2,4-dichlorophenol, 5-amino-6-chloro-o-cresol, 5-amino-4-chloro-o-cresol, hydroxybenzomorpholine, 2-amino-5-ethylphenol, 6-amino-m-cresol, 6-amino-o-cresol, 2,4-diaminophenoxyethanol, 2-amino-4-hydroxyethylaminoanisole, 1,3-bis-(2,4-diaminophenoxy)propane, 2,6-dihydroxyethylaminotoluenep, m-phenylenediamine, 2,4-diamino-1,5-di(2-hydroxyethoxybenzene, 1-naphthol, 2-methyl-1-naphthol, 1,5-naphthalenediol, 2,7-naphthalenediol, 1-acetoxy-2-methylnaphthalene, 2,6-dihydroxy-3,4-dimethylpyridine, 2,6-dimethoxy-3,5-pyridinediamine, 3-amino-6-methoxy-2-(methylamino)-pyridine, 2-amino-3-hydroxypyridine, 2,6-diaminopyridine, dihydroxyindole, 6-hydroxyindole, dihydroxyindoline, phenyl methyl pyrazolone, 1,2,4-trihydroxybenzene, 5-((2-hydroxyethyl)amino)-1,3-benzodioxol, isatin, hydroquinone, 4-formyl-1-methylquinolinium-p-toluenesulfonate and salts thereof. Preferably, the oxidative coupler dye precursors are selected from the group consisting of resorcinol, 2-methylresorcinol, 4-chlororesorcinol, 2-chlororesorcinol, m-aminophenol, hydroxybenzomorpholine, 2,4-diaminophenoxyethanol, 4-amino-2-hydroxytoluene, 2-methyl-5-hydroxyethylaminophenol, 5-((2-hydroxyethyl)amino)-1,3-benzodioxol and salts thereof.

In one embodiment, the presently claimed invention is directed to the use of at least one phenalenone for bleaching hair by generating singlet oxygen.

In one embodiment, the presently claimed invention is directed to a phenalenone which is substituted in position 2 of the phenalenone ring with one organic moiety that is substituted with at least one positively charged nitrogen atom and/or at least one neutral, protonable nitrogen atom and at least one negatively charged functional group, preferably at least one sulfonate group.

A positively charged nitrogen atom refers to a quaternary nitrogen atom, also known as an ammonium ion. This occurs when a nitrogen atom forms four covalent bonds with surrounding atoms or groups, resulting in a positively charged species.

A neutral, protonable nitrogen atom refers to a nitrogen atom that carries no net electric charge and has the capability to accept a proton (H⁺), thereby becoming positively charged.

A negatively charged functional group, also referred to as an anionic functional group, is a specific grouping of atoms within a molecule that carries a net negative electric charge due to the gain of one or more electrons. This negative charge significantly influences the chemical properties and reactivity of the molecule.

Examples of such functional groups include, but are not limited to, sulfonate groups S(═O)₂—O—, sulfate groups —O—S(═O)₂—O—, carboxylate groups C(═O)—O—, and phosphate groups PO₄³⁻.

Preferably, the organic moiety that is substituted with at least one positively charged nitrogen atom and/or at least one neutral, protonable nitrogen atom and at least one negatively charged functional group is selected from the group which consists of saturated or unsaturated alkyl radicals, saturated or unsaturated heteroalkyl radicals, saturated or unsaturated cycloalkyl radicals, saturated or unsaturated heterocyclic alkyl radicals, aryl radicals and heteroaryl radicals. The aryl radicals in each case preferably have at most 4, further preferably at most 3, further preferably at most 2, annulated rings. The aryl radicals still further preferably have 1 ring in each case. The heteroaryl radicals in each case preferably have at most 4, further preferably at most 3, further preferably at most 2, annulated rings. The heteroaryl radicals still further preferably have 1 ring in each case.

In a further preferred embodiment, the heteroaryl radicals in each case have 5 to 20, preferably 5 to 13, further preferably 5 to 7, ring atoms which comprise at least 1 carbon atom and 1 to 4 nitrogen atoms as well as optionally 1 or 2 oxygen or sulfur atoms. In a further preferred embodiment, the saturated or unsaturated heterocyclic alkyl radicals in each case have 5 to 20, preferably 5 to 13, further preferably 5 to 7, ring atoms which comprise at least 1 carbon atom and 1 to 4 nitrogen atoms as well as optionally 1 or 2 oxygen or sulfur atoms.

In a further preferred embodiment, the saturated or unsaturated alkyl radicals are in each case selected from the group which consists of methyl, ethyl, n-propyl, n-butyl, n-pentyl, n-hexyl, n-heptyl and n-octyl.

In a preferred embodiment a phenalenone which is substituted in position 2 of the phenalenone ring with one organic moiety that is substituted with at least one positively charged nitrogen atom and/or at least one neutral, protonable nitrogen atom and at least one negatively charged functional group, preferably at least one sulfonate group is selected from the group consisting of
4-[dimethyl-[(1-oxophenalen-2-yl)methyl]ammonio]butane-1-sulfonate
sodium [(1-oxophenalen-2-yl)methylamino]methane sulfonate
sodium 4-[(1-oxophenalen-2-yl)methylamino]butane-1-sulfonate
sodium 2-[(1-oxophenalen-2-yl)methylamino]ethane sulfonate
sodium 3-[(1-oxophenalen-2-yl)methylamino]propane-1-sulfonate
4-[diethyl-[(1-oxophenalen-2-yl)methyl]ammonio]butane-1-sulfonate
4-[4-[(E)-(1-oxophenalen-2-yl)methyliminomethyl]pyridin-1-ium-1-yl]butane-1-sulfonate
4-[3-methyl-2-[(1-oxophenalen-2-yl)methylamino]imidazol-1-ium-1-yl]butane-1-sulfonate and
4-[2-[(1-oxophenalen-2-yl)methylamino]thiazol-3-ium-3-yl]butane-1-sulfonate.

The presently claimed invention is associated with one or more of the following advantages.
The hair shows a lower level of off-tone, when the bleaching medium of the presently claimed invention is used, compared to conventionally bleached hair.
The hair is more efficiently bleached by the bleaching medium of the presently claimed invention.
The level of damage to the hair is similar or less, when the bleaching medium of the presently claimed invention is used.
The hair feels softer, when the bleaching medium of the presently claimed invention is used, compared to conventionally bleached hair.

In the following, there is provided a list of embodiments to further illustrate the present disclosure without intending to limit the disclosure to the specific embodiments listed below.

### EMBODIMENTS

1. A bleaching medium comprising
   (A) at least one photosensitizer which is capable of generating singlet oxygen,
   (B) at least one persulfate salt, and
   (C) at least one alkalizing agent and
   (E) at least one hydrogen peroxide source.
2. The bleaching medium according to embodiment 1, wherein the at least one photosensitizer which is capable of generating singlet oxygen (A) is selected from the group consisting of phenalenones, curcumins, flavins, riboflavins, phenoxazines, phenothiazines, phthalocyanines, naphthalocyanines, xanthenes, chlorophyll A, chlorophyll B, porphyrines, coumarins, pyrenes, perylenes, acridine orange and tetrapyrroles.
3. The bleaching medium according to embodiment 2, wherein the phenalenones are selected from phenalenones which are substituted with at least one organic moiety that is substituted with at least one positively charged nitrogen atom and at least one negatively charged functional group.
4. The bleaching medium according to embodiment 2, wherein the phenoxazine is nile blue.
5. The bleaching medium according to embodiment 2, wherein the phenothiazines are selected from the group consisting of methylene blue, toluidine blue, 1,9-dimethyl methylene blue and methylene green.
6. The bleaching medium according to embodiment 2, wherein phthalocyanines are selected from the group consisting of zinc phthalocyanine, aluminium phthalocyanine, zinc phthalocyanine tetrasulfonate and tetrakis(p-trimethylammonium)phthalocyanine zinc chloride.
7. The bleaching medium according to embodiment 2, wherein the xanthenes are selected from the group consisting of pyronine G, eosine B, eosine Y and rose Bengal.
8. The bleaching medium according to embodiment 2, wherein the porphyrines are selected from the group consisting of 5,10,15,20-tetrakis(1-methyl-4-pyridinio)porphyrine-tetra(p-toluenesulfonate) and tetrakis(p-trimethylammoniumphenyl) porphyrine chloride.
9. The bleaching medium according to embodiment 2, wherein the tetrapyrroles are selected from the group consisting of chlorin, chlorin e6 and bacteriochlorin.
10. The bleaching medium according to any one of the preceding embodiments, wherein the at least one photosensitizer which is capable of generating singlet oxygen (A) generates singlet oxygen, when exposed to electromagnetic radiation having a wavelength in the range of ≥230 nm to ≤1000 nm, preferably in the range of ≥380 nm to ≤1000 nm.
11. The bleaching medium according to any one of the preceding embodiments, wherein the at least one component (A) is present in an amount in the range of ≥0.01 wt. % to ≤10.0 wt. %, preferably in an amount in the range of ≥0.1 wt. % to ≤8.0 wt. %, more preferably in an amount in the range of ≥0.3 wt. % to ≤5.0 wt. %, most preferably in an amount in the range of ≥0.5 wt. % to ≤3.0 wt. %, based on the total weight of the bleaching medium.
12. The bleaching medium according to any of the preceding embodiments, wherein the at least one persulfate salt (B) is selected from the group consisting of ammonium persulfate, sodium persulfate and potassium persulfate.
13. The bleaching medium according to any of the preceding embodiments, wherein the at least one component (B) is present in an amount in the range of ≥3.0 wt. % to ≤30 wt. %, preferably in the range of ≥5.0 wt. % to ≤25 wt. %, more preferably in the range of ≥7.0 wt. % to ≤20 wt. %, based on the total weight of the bleaching medium.
14. The bleaching medium according to any of the preceding embodiments, wherein the at least one alkalizing agent (C) is selected from the group consisting of ammonia, alkanolamines and inorganic alkalizing agent.
15. The bleaching medium according to claim 14, wherein the alkanolamines are selected from the group consisting of monoethanolamine, 3-aminopropan-1-ol, 4-aminobutan-1-ol, 5-aminopentan-1-ol, 1-aminopropan-2-ol, 2-amino-1-propanol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol and 2-amino-2-methylpropan-1,3-diol.
16. The bleaching medium according to claim 14, wherein the inorganic alkalizing agents are selected from the group consisting of sodium hydroxide, potassium hydroxide, calcium hydroxide, barium hydroxide, sodium phosphate, potassium phosphate, alkali metal silicates, alkali metal metasilicate, alkaline earth metal silicates, alkaline earth metal metasilicate, sodium carbonate and potassium carbonate.
17. The bleaching medium according to claim 16, wherein the inorganic alkalizing agents are selected from the group consisting of sodium silicate and sodium metasilicate.
18. The bleaching medium according to any of the preceding embodiments, wherein the at least one component (C) is present in an amount in the range of ≥3.0 wt. % to ≤20 wt. %, preferably in the range of ≥4.0 wt. % to ≤15 wt. %, preferably in the range of ≥5.0 wt. % to ≤12 wt. %, based on the total weight of the bleaching medium.
19. The bleaching medium according to any of the preceding embodiments, wherein the at least one hydrogen peroxide source (E) is selected from the group consisting of hydrogen peroxide, calcium peroxide, urea peroxide, perborates and percarbonates.
20. The bleaching medium according to embodiment 19, wherein the at least one hydrogen peroxide source (E) is hydrogen peroxide, which is preferably present in the range of ≥1.0 wt. % to ≤12 wt. %, preferably in the range of ≥2.0 wt. % to ≤6.0 wt. %, based on the total weight of the bleaching medium.
21. The bleaching medium according to any of the preceding embodiments, wherein the bleaching medium comprises at least one additive (D) selected from the group consisting of sources of carbonate ions or hydrogen carbonate ions or carbamate ions, aluminosilicates, surfactants, chelants, thickening agents, fillers, amino acids, hydrolyzed proteins, fatty substances, saturated acyclic terpanes having 10 to 40 carbon atoms, C3-C20 monocarboxylic acids and C3-C10 di- or tricarboxylic acids.
22. The bleaching medium according to embodiment 21, wherein the at least one additive (D) is present in an amount in the range of ≥2.0 wt. % to ≤30 wt. %, preferably in the range of ≥3.0 wt. % to ≤25 wt. %, more preferably in the range of ≥4.0 wt. % to ≤20 wt. %, even more preferably in the range of ≥4.0 wt. % to ≤18 wt. %, most preferably in the range of ≥4.0 wt. % to ≤17 wt. %, in particular in the range of ≥4.0 wt. % to ≤15 wt. %, based on the total weight of the bleaching medium.
23. The bleaching medium according to embodiment 21, wherein the sources of carbonate ions or hydrogen carbonate ions or carbamate ions is selected from the group consisting of sodium, potassium, guanidine, arginine, lithium, calcium, magnesium, barium, ammonium salts of carbonate, carbamate and hydrogen carbonate ions.
24. The bleaching medium according to any one of embodiments 21 to 23, wherein the at least one source of carbonate ions or hydrogen carbonate ions or carbamate ions is present in an amount in the range of ≥1.0 wt. % to ≤10.0 wt. %, preferably in the range of ≥2.0 wt. % to ≤8.0 wt. %, based on the total weight of the bleaching medium.
25. The bleaching medium according to embodiment 21, wherein the aluminosilicate is sodium aluminosilicate.
26. The bleaching medium according to any one of embodiments 21 to 25, wherein the aluminosilicates are present in an amount in the range of ≥3.0 wt. % to ≤15 wt. %, preferably in the range of ≥4.0 wt. % to ≤12 wt. %, more preferably in the range of ≥4.0 wt. % to ≤10.0 wt. %, based on the total weight of the bleaching medium.
27. The bleaching medium according to any one of embodiments 21 to 26, wherein the surfactants are selected from the group consisting of alkali metal salts and alkaline earth metal salts of laurate, myristate, palmitate, stearate or behenate.
28. The bleaching medium according to any one of embodiments 21 to 26, wherein the surfactants are selected from the group consisting of sodium salts and potassium salts of laurate, palmitate or stearate.
29. The bleaching medium according to any one of embodiments 21 to 28, wherein the surfactants are present in an amount in the range of ≥1.0 wt. % to ≤9.0 wt. %, preferably in the range of ≥1.0 wt. % to ≤5.0 wt. %, more preferably in the range of ≥1.0 wt. % to ≤4.0 wt. %, based on the total weight of the bleaching medium.
30. The bleaching medium according to any one of embodiments 21 to 29, wherein the chelants are selected from the group consisting of ethylenediaminetetraacetic acid (EDTA), ethylenediamine-N,N′-disuccinic acid (EDDS), ethylenediamine-N,N′-diglutaric acid (EDDG), 2-hydroxypropylenediamine-N,N′-disuccinic acid (HPDDS), N,N-dicarboxymethylglutamic acid (GLDA), glycinamide-N,N′-disuccinic acid (GADS), ethylenediamine-N—N′-bis(ortho-hydroxyphenyl acetic acid) (EDDHA), dimethyl glucamine (DMG), N-(1-carboxyethyl)iminodiacetic acid, methylglycine N,N-diacetic acid, diethylenetriamine pentaacetic acid (DTPA), ethylenedicysteic acid (EDC), N,N′-bis(2-hydroxybenzyl)ethylenediamine-N,N′-diacetic acid (HBED), ethylene diamine tri(methylene phosphonate), tetramethylethylenediamine (TMEDA), N,N,N′,N′-tetraethylethylenediamine (TEEDA), and their salts thereof.
31. The bleaching medium according to embodiment 30, wherein the chelants are selected from the group consisting of ethylenediaminetetraacetic acid (EDTA), ethylenediamine-N,N′-disuccinic acid (EDDS), and their salts thereof.
32. The bleaching medium according to any one of embodiments 21 to 31, wherein the chelants are present in an amount in the range of ≥0.1 wt. % to ≤3.0 wt. %, preferably in the range of ≥0.2 wt. % to ≤2.0 wt. %, more preferably in the range of ≥0.3 wt. % to ≤1.5 wt. %, based on the total weight of the bleaching medium.
33. The bleaching medium according to any one of embodiments 21 to 32, wherein the thickening agents are selected from the group consisting of cellulose derivatives, a polyacrylic acids, acrylate copolymers, polysaccharide gums, xanthan gums, starch, guars, starch derivatives, alginic acid and acrylic acid-diallyldimethylammonium polymers.
34. The bleaching medium according to embodiment 33 wherein the thickening agents are selected from the group consisting of xanthan gums, starch, and alginic acid.
35. The bleaching medium according to any one of embodiments 21 to 34, wherein the thickening agents are present in an amount in the range of ≥0.5 wt. % to ≤5.0 wt. %, preferably in the range of ≥0.5 wt. % to ≤6.0 wt. %, more preferably in the range of ≥1.0 wt. % to ≤4.0 wt. %, based on the total weight of the bleaching medium.
36. The bleaching medium according to any one of embodiments 21 to 35, wherein the fillers are selected from the group consisting of kaolin clay, smectite clay and a sepiolite clay.
37. The bleaching medium according to any one of embodiments 21 to 36, wherein the fillers are present in an amount in the range of ≥0.5 wt. % to ≤10.0 wt. %, preferably in the range of ≥1.0 wt. % to ≤8.0 wt. %, more preferably in the range of ≥1.0 wt. % to ≤6.0 wt. %, based on the total weight of the bleaching medium.
38. The bleaching medium according to any one of embodiments 21 to 37, wherein the amino acids are selected from the group consisting of alanine, glycine, phenylalanine, leucine, valine, isoleucine, glutamic acid, aspartic acid, citrulline, histidine, arginine, lysine and tyrosine.
39. The bleaching medium according to any one of embodiments 21 to 38, wherein the amino acids are present in an amount in the range of ≥0.05 wt. % to ≤2.0 wt. %, preferably in the range of ≥0.1 wt. % to ≤1.5 wt. %, more preferably in the range of ≥0.1 wt. % to ≤1.3 wt. %, even more preferably in the range of ≥0.1 wt. % to ≤1.0 wt. %, based on the total weight of the bleaching medium.
40. The bleaching medium according to any one of embodiments 21 to 39, wherein the hydrolyzed proteins are selected from the group consisting of hydrolyzed keratin and hydrolyzed vegetable protein.
41. The bleaching medium according to any one of embodiments 21 to 40, wherein the C3-C10 di- or tricarboxylic acids are selected from the group consisting of tartronic acid, succinic acid, malic acid, maleic acid, citric acid, tartaric acid, adipic acid, glutaric acid, oxalic acid, sebacic acid, glyoxylic acid, malonic acid and hydroxyglutaric acid.
42. The bleaching medium according to any one of embodiments 21 to 41, wherein the C3-C20 monocarboxylic acids or C3-C10 di- or tricarboxylic acids are each present in an amount in the range of ≥0.05 wt. % to ≤2.0 wt. %, preferably in the range of ≥0.1 wt. % to ≤1.5 wt. %, more preferably in the range of ≥0.1 wt. % to ≤1.3 wt. %, even more preferably in the range of ≥0.1 wt. % to ≤1.0 wt. %, based on the total weight of the bleaching medium.
43. The bleaching medium according to any one of embodiments 21 to 42, wherein the saturated acyclic terpanes having 10 to 40 carbon atoms are selected from the group consisting of squalane, isosqualane and neosqualane.
44. The bleaching medium according to any one of embodiments 1 to 43 comprising
   (A) ≥0.5 wt. % to ≤3.0 wt. % of at least one photosensitizer which is capable of generating singlet oxygen,
   (B) ≥3.0 wt. % to ≤30 wt. % of at least one persulfate salt,
   (C) ≥20 wt. % to ≤32 wt. % of at least one alkalizing agent, and
   (D) ≥2.0 wt. % to ≤25 wt. % of at least one additive (D) selected from the group consisting of sources of carbonate ions or hydrogen carbonate ions or carbamate ions, aluminosilicates, surfactants, chelants, thickening agents, fillers, amino acids, hydrolyzed proteins, fatty substances, saturated acyclic terpanes having 10 to 40 carbon atoms, C3-C20 monocarboxylic acids and C3-C10 di- or tricarboxylic acids.
45. A method for bleaching hair comprising at least the steps of:
   a) applying the bleaching medium according to any one of embodiments 1 to 44 to hair to obtain covered hair,
   b) exposing the covered hair to electromagnetic radiation having a wavelength in the range of ≥230 nm to ≤1000 nm, preferably in the range of ≥380 nm to ≤1000 nm,
   c) rinsing the bleaching medium from the covered hair and
   d) optionally drying the bleached hair.
46. The method according to embodiment 45, wherein in step b) the covered hair is exposed to electromagnetic radiation for a period in the range of ≥1 minute to ≤50 minutes.
47. Use of at least one compound selected from the group consisting of phenalenones, curcumins, flavins, riboflavins, phenoxazines, phenothiazines, phthalocyanines, naphthalocyanines, xanthenes, chlorophyll A, chlorophyll B, porphyrines, coumarins, pyrenes, perylenes, acridine orange and tetrapyrroles for bleaching hair by generating singlet oxygen.
48. The use according to embodiment 47, wherein the at least one compound is selected from phenalenones.
49. A phenalenone which is substituted in position 2 of the phenalenone ring with one organic moiety that is substituted with at least one positively charged nitrogen atom and at least one negatively charged functional group.

While the presently claimed invention has been described in terms of its specific embodiments, certain modifications and equivalents will be apparent to those skilled in the art and are intended to be included within the scope of the presently claimed invention.

### EXAMPLES

The presently claimed invention is illustrated in detail by non-restrictive working examples which follow. More particularly, the test methods specified hereinafter are part of the general disclosure of the application and are not restricted to the specific working examples.

### Materials

Potassium persulfate commercially available from United Initiators, Pullach, Germany
Alkaline sodium silicate commercially available from BASF, Ludwigshafen/Rhine, Germany
Ammonium persulfate commercially available from United Initiators, Pullach, Germany
Magnesium carbonate hydroxide commercially available from Dr. Paul Lohmann, Emmerthal, Germany
Sodium stearate commercially available from Matrix Chemie
Squalane commercially available from Aprinnova
Xanthan gum commercially available from KP Kelco, GroBenbrode, Germany
Algin commercially available from BASF, Ludwigshafen/Rhine, Germany
Disodium EDTA commercially available from BASF, Ludwigshafen/Rhine, Germany
D,L-Malic acid commercially available from Merck Chemicals GmbH, Darmstadt, Germany
Mineral oil commercially available from Panama PetroChem Ltd., India

### Synthesis of Phenalenone

### 1) Synthesis of Phenalenone (IV):

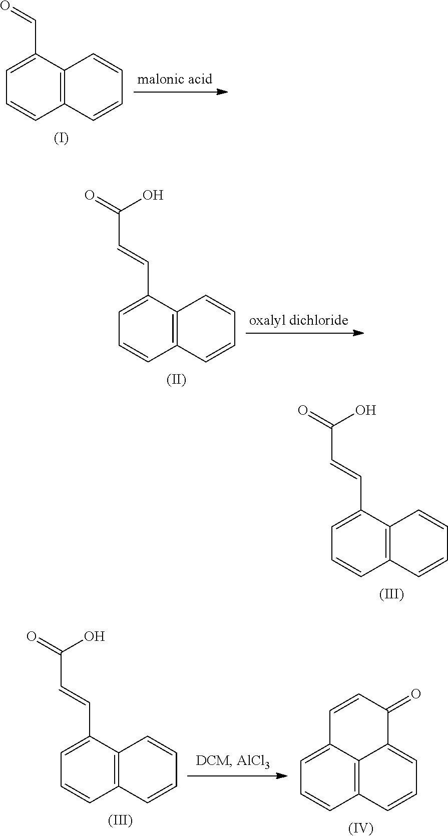

Malonic acid (4.0 eq., 53.3 g, 512 mmol) and 1-naphthaldehyde (I) (1.0 eq., 20.0 g, 128 mmol, 17.4 mL) were dissolved in pyridine (70 mL, 3.5 parts per volume) at room temperature while adding catalytic amounts of piperidine as base (0.2 eq., 2.2 g, 26 mmol, 2.5 mL). The reaction mixture was then heated to reflux for 2 h. The reaction mixture was allowed to cool down to room temperature, followed by the addition of 250 mL iced water under vigorous stirring while a white-beige residue precipitated. The yield was further increased while adjusting the pH level to 4 with conc. hydrochloric acid. The residue was collected by vacuum filtration and washed with iced water to obtain the intermediate (E)-3-(1-naphthyl)prop-2-enoic acid (II) as slightly beige powder in 93% yield (23.9 g, 119 mmol).

(E)-3-(1-naphthyl)prop-2-enoic acid (II) (1.0 eq., 3.0 g, 15 mmol) was suspended in dichloromethane (DCM) (30 mL, 10 parts per volume), catalytic amounts of DMF (100 μl) were added and the mixture was cooled to 0° C. Then, oxalyl chloride (1.5 eq., 2.9 g, 23 mmol, 2.0 mL) was slowly added and once the addition was complete, the reaction mixture was heated to room temperature under stirring until a clear, yellow solution was formed. The reaction mixture was again cooled to 0° C. while adding then in one portion aluminum chloride (2.0 eq., 4.0 g, 30 mmol). The reaction mixture was slowly heated to room temperature and further heated to reflux for 4 h. After cooling down to room temperature, the reaction mixture was filtered, and the solvent was completely evaporated under reduced pressure. The resulting residue was collected in 50 mL saturated sodium bicarbonate solution and stirred for 30 min. The formed precipitate was collected via filtration and washed with water and dried. The crude product was recrystallized in toluene to obtain pure phenalenone (IV) in 77% yield (2.1 g, 12 mmol).

### 2) Synthesis of Substituted Phenalenone

Phenalenone was converted into 2-(chloromethyl)phenalen-1-one according to the protocol described in Godard et al., ACS Omega 2020, 5, 28264-28272.

### 2a) Synthesis of 4-[dimethyl-[(1-oxophenalen-2-yl)methyl]ammonio]butane-1-sulfonate (VI)

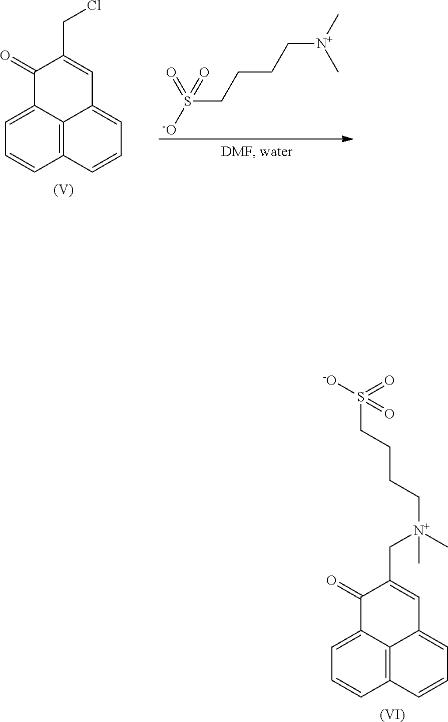

0.23 g (1.00 mmol) 2-(chloromethyl)phenalen-1-one (V) was dissolved in 2 mL DMF to form a clear yellow solution and heated to 85° C. 0.90 g (5.00 mmol) 4-(dimethylammonio)butane-1-sulfonate was dissolved in a separate vessel in 3 mL DMF and 5 mL water. 4-(dimethylammonio)butane-1-sulfonate was sensitive to light, hence, the course of the reaction was continued in the dark. The solution containing the 4-(dimethylammonio)-butane-1-sulfonate was slowly added at 85° C. to the 2-(chloromethyl)phenalen-1-one (V) solution in DMF. The reaction mixture was then further heated to 100° C. under stirring for 24 h forming an intense dark yellow solution. The reaction mixture was then cooled to room temperature and diluted with 10 mL DMF. The excess of 4-(dimethylammonio)butane-1-sulfonate precipitates from the solution and was removed via filtration, followed by washing the filter cake with small amounts of DMF. The filtrate was then evaporated under reduced pressure to a volume of ca. 10% and cold diethyl ether was added. The precipitated crude product was collected by filtration and further recrystallized with methanol to remove more polar impurities to yield the desired 4-[dimethyl-[(1-oxophenalen-2-yl)methyl-]ammonio]butane-1-sulfonate (VI) in 50% yield (0.19 g).

### 2b) Synthesis of 4-[(1-oxophenalen-2-yl)methylamino]butane-1-sulfonic acid (VIII)

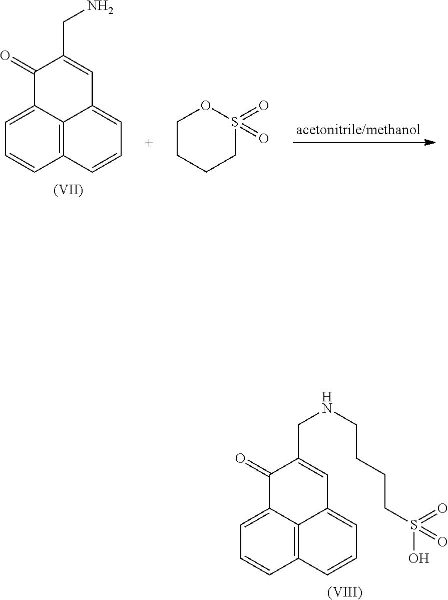

0.15 g (0.72 mmol) 2-(aminomethyl)phenalen-1-one (VII) were dissolved in a mixture comprising 4 mL acetonitrile and 1 mL methanol at 50° C. to form a clear reddish solution. 0.10 g (0.75 mmol) oxathiane 2,2-dioxide was added to this solution at 50° C. The reaction mixture was then further heated to 70° C. for 12 h. The solvent was evaporated under reduced pressure and the formed precipitated was then collected with 5 mL diethyl ether and filtrated. The residue was then washed with 5 mL toluene to obtain the desired 4-[(1-oxophenalen-2-yl)methylamino]butane-1-sulfonic acid (VIII) in 53% yield (0.13 g).

### 2c) Synthesis of sodium; [(1-oxophenalen-2-yl)methylamino]methane sulfonate (IX)

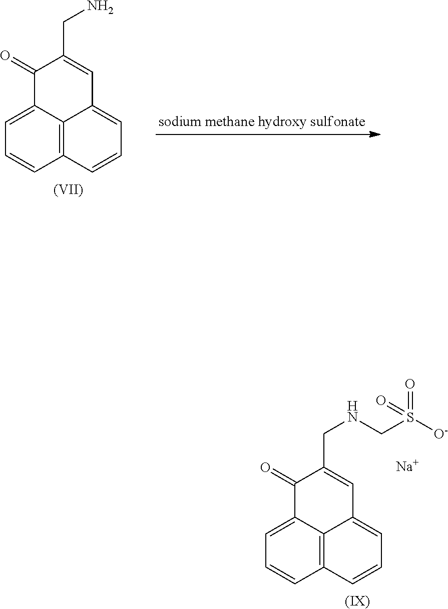

0.56 g (2.29 mmol) 2-(aminomethyl)phenalen-1-one (VII) were dissolved in 10 mL methanol. In a separate vessel, 0.355 g (2.51 mmol) commercial sodium methane hydroxy sulfonate were dissolved in 1 mL water. This solution was added at 60° C. to the solution of 2-(aminomethyl)phenalen-1-one (VII) in methanol. The reaction mixture was then stirred at 60° C. for further 2 h. The solvent was then carefully evaporated to obtain a dark red slurry, followed by the addition of 5 mL water, and stirred at room temperature. The reaction mixture was then heated to 60° C. followed by the addition of 1 mL ethyl acetate. The formed yellowish-orange crystals precipitated from the solution and were collected by filtration. The crystals were grounded in a mortar by addition of water and acetone. The formed solid was collected by filtration while another portion of crystals precipitated from the filtrate. The collected solids were combined to yield the desired compound sodium; [(1-oxophenalen-2-yl)methylamino]methane sulfonate (IX) in 63% yield (0.47 g).

### Hair Tresses

Caucasian hair switches of Level 7.0 and Level 5.0 in the form of 10 cm long and 1 cm wide tresses supplied by Kerling International Haarfabrik GmbH, Backnang, Germany.

### Test Methods

### Combing Force Measurement

Various external influences, such as certain cosmetic treatments (bleaching, colouring, permanent waving), exposure to the weather, frequent combing and brushing affect the combing ease due to a degradation of the hair cuticle. Thus, the combing ease of hair is a major parameter for describing the quality/degree of damage of hair on the one hand and/or the effectiveness of conditioning respectively hair repair treatments on the other hand. The principle of most methods for the determination of combing properties consists in measuring the force to pull a comb through a hair tress under definite conditions (Ref.: C. R. Robbins, Chemical and Physical Behavior of Human Hair, 5th Edition, p. 646 ff., Springer-Verlag, Berlin Heidelberg 2012. ISBN 978-3-642-25610-3). For this test, an automated device was used in which the tresses to be tested are taken from a storage chamber and are fixed to a force meter. The tresses were combed automatically at constant speed. For each combing stroke the combing force was recorded as a function of the distance combed through (length of the tress). For comparison purposes 20 combing strokes per tress on at least 3 tresses per product sample were averaged. The Wet Combing Force was the average force along the tress. The lower the combing force the better the combing ease.

### Hunter Lab Colour Measurement

The L*a*b* color values obtained in the foregoing examples were determined with a Chromameter II color meter supplied by Minolta. The L value stands for brightness (namely the higher the L-value the higher is lift), the a-value being a measure of the red content (namely, the higher the a-value, the higher is the red content). The b-value is a measure of the blue content of the color, the blue content being the higher the more negative the b-value.

### FT-IR Damage Measurement

Damage caused to the hair was assessed by a FTIR (Fourier Transform Infrared) method, which was established to be suitable for studying the effects of keratin surface damage. Strassburger, J., J. Soc. Cosmet Chem., 36, 61-74 (1985); Joy, M. & Lewis, D. M., Int. J. Cosmet. Sci., 13, 249-261 (1991); Signori, V. and Lewis, D. M., Int. J. Cosmet. Sci., 19, 1-13 (1997)). In particular, these authors have shown that the method was suitable for quantifying the amount of cysteic acid. In general, the oxidation of cystine is thought to be a suitable marker by which to monitor the overall oxidation of the keratinous part of the fiber. Net, the measurement of cysteic acid units by FT-IR was commonly used.

Signori and Lewis (D. M., Int. J. Cosmet. Sci., 19, 1-13 (1997)) showed that FT-IR using a diamond Attenuated Total Internal Reflection (ATR) cell was a sensitive and reproducible way of measuring the cysteic acid content of single fibers and bundles. Hence, the method that was employed to measure the cysteic acid content of multiple fibre bundles and full hair switches, was based upon the FTIR diamond cell ATR method employed by Signori and Lewis (1997). The detailed description of the method for testing the different damage inhibitors follows thereafter:

A Perkin Elmer Spectrum® 1 Fourier Transform Infrared (FTIR) composition equipped with a diamond Attenuated Total Internal Reflection (ATR) cell was used to measure the cysteic acid concentration in mammalian or synthetic hair. In this method, hair switches of various sizes and colours were used. The switches were platted (~1 plait per cm) in order to minimize variations in surface area of contact between readings. The bleaching method described below was repeated for 5 cycles to mimic the behaviour of hair after repeated bleaching cycles. Following this treatment, four readings per switch were taken (⅓ and ⅔s down the switch on both sides), and an average calculated. Backgrounds were collected every 4 readings, and an ATR cell pressure of 1 N/m was employed. The cell was cleaned with ethanol between each reading, and a contamination check was performed using the monitor ratio mode of the instrument. As prescribed by Signori &amp; Lewis in 1997, a normalized double derivative analysis routine was used. The original spectra was initially converted to absorbance, before being normalized to the 1450 cm⁻¹ band (the characteristic and invariant protein CH₂ stretch). This normalized absorbance was then twice derivatised using a 13-point averaging. The value of the 1450 cm⁻¹ normalized 2nd derivative of the absorbance at 1040 cm⁻¹ was taken as the relative concentration of cysteic acid. This figure was multiplied by −1×10⁴ to recast it into suitable units.

### Formulations

The bleach powder formulation samples were prepared by mixing all the dry ingredients together for 5 minutes inside a mixer where the rotational speed of the mixing blade was 3.5 rpm. Dedusting component was in a drop by drop manner while the rotational speed was set at 3 rpm and finally the speed was increased to 3.5 rpm for 6 minutes which ensured a homogenous mixture.

TABLE 1
Bleach powder formulation

| | Powder 1 in wt.-% | Powder 2 in wt.-% |
|---|---|---|
| Potassium persulfate | 34.00 | 34.00 |
| Alkaline sodium silicate | 17.00 | 17.00 |
| Ammonium persulfate | 10.00 | 10.00 |
| Magnesium carbonate | q.s. | q.s. |
| hydroxide | | |
| Sodium stearate | 4.00 | 4.00 |
| Xanthan gum | 2.80 | 2.80 |
| Algin | 0.50 | 1.00 |
| Disodium EDTA | 1.30 | 1.30 |
| Mineral oil | 5.00 | 5.00 |

TABLE 2
Oxidative composition example (6% Developer)

| | Developer 1 |
|---|---|
| Ingredients | (% w/w) |
| Water Purified (q.s) | q.s. |
| Salicylic Acid | 0.10 |
| Disodium Phosphate | 0.08 |
| Phosphoric Acid (85%) | 0.06 |
| Etidronic Acid | 0.01 |
| Hydrogen peroxide (50% Hydroxide | 12.00 |
| peroxide Interox co-SO SOLVAY) | |
| Cetearyl Alcohol (BASF) | 5.00 |
| Steareth-20 (CRODA) | 0.50 |

TABLE 3
Photosensitizer formulation.
20 mg of unsubstituted phenalenone (IV) was dissolved
in the following solvents at different amounts.

| | Amount of | | δd | δp | δh | |
|---|---|---|---|---|---|---|
| | solvent | | (MPa)^{1/2} | (MPa)^{1/2} | (MPa)^{1/2} | Dissolution |
| Isopropyl myristate | 10 | g | 16.0 | 2.0 | 5.6 | Yes |
| Ethoxy diglycol | 10 | g | 16.2 | 7.5 | 12.3 | Yes |
| 2-Methoxyethanol | 10 | g | 15.1 | 9.4 | 12.6 | Yes |
| 1-Methyl-2-pyrrolidone | 500 | mg | 18.0 | 12.3 | 7.2 | Yes |
| DMSO | 500 | mg | 18.4 | 16.4 | 10.2 | Yes |
| Propylene glycol | 10 | g | 16.8 | 11.4 | 26.4 | No |
| Ethylene glycol | 10 | g | 17.0 | 11.0 | 26.0 | No |
| Hexylene glycol | 2 | g | 16.1 | 6.1 | 13.6 | No |
| Isopropyl alcohol | 2 | g | 15.8 | 6.1 | 16.4 | No |

TABLE 4
Tint composition

| | Tint 1 | Tint 2 |
|---|---|---|
| Tint Ingredients | (% w/w) | (% w/w) |
| DI water | q.s | q.s |
| Disodium EDTA (BASF) | 0.100 | 0.100 |
| Ascorbic acid (OSKAR BERG) | 0.300 | 0.300 |
| Sodium Sulfite (BCD CHEMIE) | 0.400 | 0.400 |
| Ammonia Solution (25%) (Brenntag GmbH) | 18.500 | 18.500 |
| Ammonium Sulfate | 1.450 | 1.450 |
| Sodium, Hydroxide | x | x |
| 1-Hydroxyethyl 4,5-Diamino Pyrazole Sulfate | x | x |
| 2-Methoxymethyl-p-Phenylenediamine | x | x |
| Hydroxyethyl-3,4-Methylenedioxyaniline HCl | x | x |
| 2,4-Diaminophenoxyethanol HCl | x | x |
| 4-Amino-2-Hydroxytoluene | x | x |
| Trisodium Ethylenediamine Disuccinate | x | x |
| Xanthan Gum | x | x |
| Propylene Glycol | 8.000 | 8.000 |
| Cetearyl Alcohol (BASF) | (10.000) | (10.000) |
| Dicetyl Phosphate AND Ceteth-10 Phosphate | X | x |
| Steareth-200 | x | x |
| Alanine | 0.250 | 0.250 |

### Bleaching Method

In the examples of the presently claimed invention either Welloxon® Perfect 6%, or 9% oxidizing formula containing 6 wt. % or 9 wt. % hydrogen peroxide in an aqueous medium, based on the overall weight of the product (Developer from the Wella Germany GmbH, Darmstadt, Germany) was used.

Bleaching compositions were produced by mixing bleach powders described in Table 1 with a photosensitizer formulation and with a market developer, as described above.

The following protocol was used to assess the performance of each oxidative hair treatment composition.
1. Three natural Caucasian hair tresses were used as described above
2. Apply a bleaching composition prepared using Welloxon® Perfect 9% and Blondor®.
3. 1 part of each bleach powder was mixed with 1.5 parts of oxidizing composition (Welloxon® Perfect 9%) and then applied and worked throughout the hair tresses with a brush. 4 g of product was applied to each 1 g tress.
3. The tresses were irradiated with electromagnetic radiation having a frequence of 415 nm and an energy density of 35 mW/cm² or put into an oven at 30° C. for 40 minutes.
4. The tresses were then rinsed in water, 4 L min⁻¹ at a temperature of 37+−2° C. for 2 minutes
5. The tresses are combed twice (rough & fine side of comb) and placed inside between pre-wettened paper sheets.
6. Combing Force measurements were performed 3 times for each tress.

### EXAMPLES

| | Hair | Energy | Bleach | | | | | |
|---|---|---|---|---|---|---|---|---|
| | tresses | source | powder | Developer | Photosensitizer | Concentration | ΔFTIR | ΔL |
| 1 * | 5.0 | Heat | 1 | 1 | — | — | 109.61 | 36.3245 |
| 2 * | 5.0 | Radiation | 1 | 1 | — | — | 103.11 | 36.6825 |
| 3 | 5.0 | Radiation | 1 | 1 | Phenalenone | 2.0 | 113.24 | 37.7135 |
| | | | | | (IV) | | | |
| 4 | 5.0 | Radiaton | 1 | 1 | Phenalenone | 2.0 | 118.91 | 43.6275 |
| | | | | | (IX) | | | |
| 5 * | 7.0 | Heat | 1 | 1 | — | — | 111.53 | 40.208 |
| 6 * | 7.0 | Radiation | 1 | 1 | — | — | 105.65 | 38.7225 |
| 7 | 7.0 | Radiation | 1 | 1 | Phenalenone | 2.0 | 110.05 | 41.949 |
| | | | | | (IV) | | | |
| 8 | 7.0 | Radiaton | 1 | 1 | Phenalenone | 2.0 | 111.61 | 42.6025 |
| | | | | | (IX) | | | |

* outside the scope of the presently claimed invention

The provided examples demonstrate that the inclusion of phenalenone, both IV and IX, in the hair treatment process results in higher lift, as indicated by the increased ΔL values, compared to treatments without phenalenone. Specifically, the examples utilizing phenalenone (examples 3, 4, 7, and 8) exhibit ΔL values ranging from 37.7135 to 43.6275, significantly higher than the control examples without phenalenone (examples 1, 2, 5, and 6) which show ΔL values between 36.3245 and 40.208. Furthermore, the FTIR measurements indicate that the damage caused, reflected by the ΔFTIR values, is comparable between the phenalenone-containing treatments and the controls. For instance, ΔFTIR values for phenalenone-treated hair range from 110.05 to 118.91, while those for controls range from 103.11 to 111.53. This demonstrates that phenalenone enhances hair lift without substantially increasing the oxidative damage to the keratin structure, as measured by the formation of cysteic acid.

1. A bleaching medium comprising
(A) at least one photosensitizer which is capable of generating singlet oxygen which is at least one phenalenone,
(B) at least one persulfate salt, and
(C) at least one alkalizing agent and
(E) at least one hydrogen peroxide source.

2. The bleaching medium according to claim 1, wherein the bleaching medium further comprises at least one second photosensitizer which is capable of generating singlet oxygen (A2) which is selected from the group consisting of curcumins, flavins, riboflavins, phenoxazines, phenothiazines, phthalocyanines, naphthalocyanines, xanthenes, chlorophyll A, chlorophyll B, porphyrines, coumarins, pyrenes, perylenes, acridine orange and tetrapyrroles.

3. The bleaching medium according to claim 1, wherein the at least one phenalenone is unsubstituted or is selected from phenalenones which are substituted, preferably which are substituted in position 2 of the phenalenone ring, with at least one organic moiety that is substituted with at least one positively charged nitrogen atom and/or at least one neutral, protonable nitrogen atom and at least one negatively charged functional group.

4. The bleaching medium according to claim 2, wherein the phenoxazine is nile blue.

5. The bleaching medium according to claim 2, wherein the phenothiazines are selected from the group consisting of methylene blue, toluidine blue, 1,9-dimethyl methylene blue and methylene green.

6. The bleaching medium according to claim 2, wherein phthalocyanines are selected from the group consisting of zinc phthalocyanine, aluminium phthalocyanine, zinc phthalocyanine tetrasulfonate and tetrakis(p-trimethylammonium)phthalocyanine zinc chloride, or wherein the xanthenes are selected from the group consisting of pyronine G, eosine B, eosine Y and rose Bengal, or wherein the porphyrines are selected from the group consisting of 5,10,15,20-tetrakis(1-methyl-4-pyridinio)porphyrine-tetra(p-toluenesulfonate) and tetrakis(p-trimethylammoniumphenyl) porphyrine chloride, or wherein the tetrapyrroles are selected from the group consisting of chlorin, chlorin e6 and bacteriochlorin.

7. The bleaching medium according to any one of the preceding claims, wherein the at least one phenalenone generates singlet oxygen, when exposed to electromagnetic radiation having a wavelength in the range of ≥230 nm to ≤1000 nm, preferably in the range of ≥380 nm to ≤1000 nm, more preferably in the range of ≥380 nm to ≤780 nm, even more preferably in the range of ≥380 nm to ≤490 nm, most preferably in the range of ≥390 nm to ≤430 nm, and/or wherein the at least one component (A) is present in an amount in the range of ≥0.01 wt. % to ≤10.0 wt. %, preferably in an amount in the range of ≥0.10 wt. % to ≤8.0 wt. %, more preferably in an amount in the range of ≥0.15 wt. % to ≤5.0 wt. %, most preferably in an amount in the range of ≥0.20 wt. % to ≤3.0 wt. %, based on the total weight of the bleaching medium, and/or wherein the at least one persulfate salt (B) is selected from the group consisting of ammonium persulfate, sodium persulfate and potassium persulfate, and/or wherein the at least one alkalizing agent (C) is selected from the group consisting of ammonia, alkanolamines and inorganic alkalizing agent, and/or wherein the at least one hydrogen peroxide source (E) is selected from the group consisting of hydrogen peroxide, calcium peroxide, urea peroxide, perborates and percarbonates, and/or wherein the bleaching medium comprises at least one additive (D) selected from the group consisting of sources of carbonate ions or hydrogen carbonate ions or carbamate ions, aluminosilicates, surfactants, chelants, thickening agents, fillers, amino acids, hydrolyzed proteins, fatty substances, saturated acyclic terpanes having 10 to 40 carbon atoms, C3-C20 monocarboxylic acids and C3-C10 di- or tricarboxylic acids.

8. The bleaching medium according to any of the preceding claims, wherein the bleaching medium comprises at least one polar solvent has Hansen solubility parameters in the range of δd≥15.0 to ≤20.0 (MPa)^{1/2}, δp≥2.0 to ≤28.0 (MPa)^{1/2} and δh≥5.0 to ≤13.0 (MPa)^{1/2}.

9. The bleaching medium according to claim 8, wherein the at least one solvent is selected from the group consisting of methyl laurate, isopropyl myristate, ethoxy diglycol, 2-methoxy ethanol, 1-methoxy-2-propanol, dimethyl sulfoxide and 1-methyl-2-pyrrolidon.

10. The bleaching medium according to claim 9, wherein the at least one solvent is present in an amount in the range of ≥0.5 wt. % to ≤5.0 wt. %, preferably in the range of ≥0.5 wt. % to ≤2.0 wt. %.

11. A method for bleaching hair comprising at least the steps of:
a) applying the bleaching medium according to any one of claims 1 to 10 to hair to obtain covered hair,
b) exposing the covered hair to electromagnetic radiation having a wavelength in the range of ≥230 nm to ≤1000 nm, preferably in the range of ≥380 nm to ≤1000 nm, more preferably in the range of ≥380 nm to ≤780 nm, even more preferably in the range of ≥380 nm to ≤490 nm, most preferably in the range of ≥390 nm to ≤430 nm,
c) rinsing the bleaching medium from the covered hair and
d) optionally drying the bleached hair, preferably wherein the electromagnetic radiation has an energy density in the range of ≥1 mW/cm² to ≤70 mW/cm², preferably in the range of ≥10 mW/cm² to ≤50 mW/cm², even more preferably in the range of ≥20 mW/cm² to ≤40 mW/cm².

12. Use of at least phenalenone for bleaching hair by generating singlet oxygen.

13. The use according to claim 12, wherein the at least one phenalenone is selected from the group consisting of unsubstituted phenalenone and phenalenones which are substituted with at least one organic moiety that is substituted with at least one positively charged nitrogen atom and/or at least one neutral, protonable nitrogen atom and at least one negatively charged functional group.

14. A phenalenone which is substituted in position 2 of the phenalenone ring with one organic moiety that is substituted with at least one positively charged nitrogen atom and/or at least one neutral, protonable nitrogen atom and at least one negatively charged functional group.

15. The phenalenone according to claim 14, wherein the at least one negatively charged functional group is a sulfonate group, and/or which is selected from the group consisting of
4-[dimethyl-[(1-oxophenalen-2-yl)methyl]ammonio]butane-1-sulfonate
sodium [(1-oxophenalen-2-yl)methylamino]methane sulfonate
sodium 4-[(1-oxophenalen-2-yl)methylamino]butane-1-sulfonate
sodium 2-[(1-oxophenalen-2-yl)methylamino]ethane sulfonate
sodium 3-[(1-oxophenalen-2-yl)methylamino]propane-1-sulfonate
4-[diethyl-[(1-oxophenalen-2-yl)methyl]ammonio]butane-1-sulfonate
4-[4-[(E)-(1-oxophenalen-2-yl)methyliminomethyl]pyridin-1-ium-1-yl]butane-1-sulfonate
4-[3-methyl-2-[(1-oxophenalen-2-yl)methylamino]imidazol-1-ium-1-yl]butane-1-sulfonate and
4-[2-[(1-oxophenalen-2-yl)methylamino]thiazol-3-ium-3-yl]butane-1-sulfonate.
